(19) United States
(12) Patent Application Publication
(10) Pub. No.: US 2009/0290719 A1
(43) Pub. Date: Nov. 26, 2009

(54) STETHOSCOPIC ASSEMBLY WITH RECORD/PLAYBACK FEATURE

(75) Inventors: Andrew J. Kugler, Albany, NY (US); Charles N. Stewart, Skaneateles, NY (US); John A. Lane, Weedsport, NY (US); Alisa Jean Robinson, Fayetteville, NY (US); Suzanne Abate Gunter, Concord, NC (US); Udaya Patnaik, San Mateo, CA (US); Michael D. Smith, San Francisco, CA (US); Lucas C. McCann, Madison, WI (US); Ashley Elizabeth Tudor, San Francisco, CA (US); Isabel P. O'Meara, Menlo Park, CA (US)

Correspondence Address: HISCOCK & BARCLAY, LLP, ONE PARK PLACE, 300 S. STATE ST., SYRACUSE, NY, 13202, US

(73) Assignee: Welch Allyn, Inc.

(21) Appl. No.: 12/125,417

(22) Filed: May 22, 2008

(51) Int. Cl. A61B 7/04

(52) U.S. Cl. 381/67

(57) ABSTRACT

A stethoscopic assembly includes a stethoscope having a chest piece interconnected to at least one earpiece, the chest piece having an acoustical sensor enabling patient sounds to be heard through the at least one earpiece. A microprocessor connected to the acoustical sensor of the stethoscope includes a buffer wherein sound data picked up from the microphone is stored in volatile memory for a predetermined time period. At least one user operable switch connected to the microprocessor enables selective capture of sound data for storage and playback, such as through at least one speaker. In some versions the acoustical sensor is releasable from the chest piece.

### FIELD OF THE INVENTION

This invention relates to the field of listening devices and more specifically to a stethoscopic assembly in which sound data picked up by a stethoscope may be transmitted to one or a plurality of receivers. A predetermined amount of buffered sound data can be selectively captured and transmitted, enabling sound data of interest to be shared, such as with a patient and/or other individuals.

### BACKGROUND OF THE INVENTION

Lack of patient involvement during examinations is a significant issue in the medical industry. Patients often submit themselves for examination, but are uncertain of their physician's level of expertise. Since the data used by the physician is often not reviewed with the patient, patients may consider a physician's diagnosis to be suspect. This sequestering of the medical data may also leave many patients feeling unconvinced of the physician's diagnosis, and uninvolved in their medical treatment. It would be advantageous if both physicians and patients could view and/or review the medical data at the same time—thus permitting physicians to both demonstrate their expertise and educate the patient. For example, if a fractured bone is suspected, when the diagnosis is given to the patient, the x-ray is used to illustrate the findings.

Moreover, when physicians are training students, it can be problematic for both physicians and students to analyze data simultaneously, since the data itself may be transient. It would be advantageous if the physician could monitor the data from a patient while the student is observing and, after a data point of interest has been detected, record the data that has just occurred. The recording may then be reviewed with the student (and patient) to further discuss the data.

It is also desirable to permit more than one physician to review patient data simultaneously. Such a configuration permits several medical practitioners, with various levels of expertise, to review received sound data and offer their input. Typically and with current stethoscopes, multiple physicians separately listen to the patient's heart and lung sounds at different moments in time with the hope that these sounds are consistent from one moment to the next. However, such consistency usually does not exist. Moreover, since the sounds can be transitory in nature, changing from one moment to the next, it is far too easy for one of the physicians to fail to hear a characteristic noise that a second physician was able to detect. Some stethoscopes have been developed that permit more than one listener to hear the bodily noises of a patient. However, in these systems the multiple listeners use earpieces which prevent conversation while listening to the patient. In addition, stethoscope systems that currently permit multiple physicians to listen to a signal simultaneously are exceptionally complex as well as expensive and are often inconvenient for use.

For the preceding reasons, among others, a stethoscope modification is desired that obviates at least some of these shortcomings.

### SUMMARY OF THE INVENTION

The invention comprises, in one form thereof, a stethoscope assembly selectively permits multiple listeners to hear patient sounds. For purposes of this discussion, “multiple’ refers to at least two listeners that can include the physician and patient and/or other physicians or others (for example, family members). Sound waves obtained from a patient are converted into corresponding sound data and then transmitted to one or more receivers. The sound data are continuously stored in the assembly in a memory buffer having a predetermined size.

Upon activation of a control switch, the switch being provided on a separable component or on the stethoscope itself, the data within the buffer is recorded and stored into memory. If the control switch is not activated before the buffer is full, the buffer is overwritten, beginning with the oldest data in the buffer. In this fashion, a medical practitioner may decide to record a sound for future reference after the sound has occurred. This allows the clinician to focus on listening (rather than if the unit records only after a record control was actuated).

One advantage of the herein described stethoscopic assembly is that both physician and patient (as well as other individuals) can readily and simultaneously hear the same patient sounds for analysis, review or other purposes. Another advantage provided herein is that the physician can selectively capture a sound of interest after the sound has occurred, since the sound has already been buffered. Therefore, buffering for later recording and playback facilitates the ability of the physician to concentrate on the task of listening to the patient rather than attempting to synchronize any capture of sound data with the physician or caregiver hoping to “find” a similar sound in the hopes that contemporaneous storage and capture can take place. After the sound(s) of interest are identified, the clinician is able to draw the patient (or other party's) attention to the sounds of interest that are normal or characteristic of a pathology.

These and other features and advantages will become readily apparent to those of sufficient skill in the field of the invention from the following Detailed Description, which should be read in conjunction with the accompanying drawings.

### BRIEF DESCRIPTION OF THE DRAWINGS

The present invention is disclosed with reference to the accompanying drawings, wherein:

FIG. 1 is a perspective view of a stethoscope assembly in accordance with one embodiment;

FIG. 2 is a schematic view of the stethoscope assembly of FIG. 1;

FIG. 3 is a schematic depiction of one embodiment of the assembly of FIGS. 1 and 2; and

FIG. 4 is a diagrammatic view of a stethoscopic assembly in accordance with another embodiment.

### DETAILED DESCRIPTION

The following description relates to a stethoscopic assembly made in accordance with certain preferred embodiments. It will be readily apparent that other modifications and variations are possible within the intended scope of the inventive concepts described herein. In addition, certain terms are used throughout in order to provide a suitable frame of reference with regard to the accompanying drawings. It is not intended, however, that these terms are to be narrowly interpreted, except where specifically indicated. In addition, similar parts are herein labeled with the same reference numerals between various drawing views for purposes of clarity.

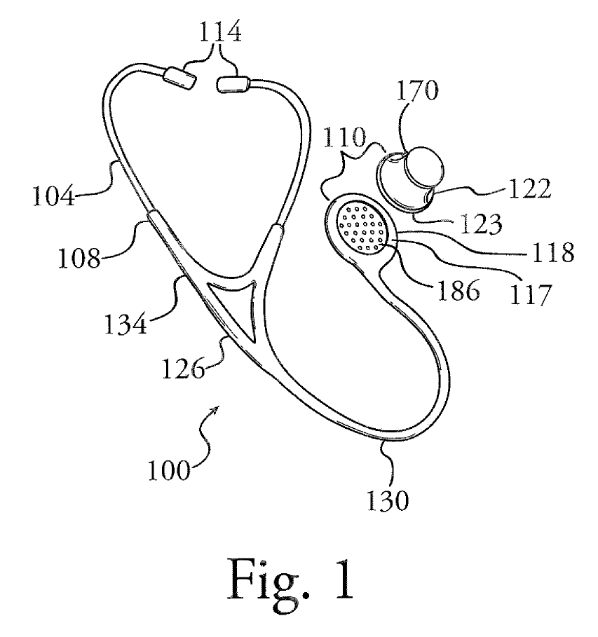

Referring to the figures and specifically to FIG. 1, a stethoscopic assembly 100 made in accordance with a preferred embodiment includes a stethoscope 104, which includes a headset 108 having a chest piece 110 that is interconnected to at least one earpiece 114. In the herein described embodiment of FIG. 1, the headset 108 includes two earpieces 114 wherein the depicted stethoscope 104 is a binaural stethoscope, such as structurally described by way of example in U.S. Pat. No. 4,254,302. Unlike the noted patent, however, the chest piece 110 of the herein described stethoscopic assembly 100 can be defined by a two-part assemblage comprising a base portion 118 and a releasably connected portion 122 as depicted according to FIG. 1. It will be readily apparent from the following discussion that this assemblage design is exemplary and a single or integral chest piece can also be utilized herein.

The earpieces 114 and the chest piece 110 of the stethoscope 104 may be interconnected by a variety of means. In the embodiment depicted in FIG. 1, the earpieces 114 are interconnected to the base portion 118 of the chest piece 110 via conventional hoses 126 that are split from a single hose 130 extending from the base portion 118 at a yoke 134 and wherein sounds from the chest piece 110 are each channeled acoustically to a corresponding earpiece 114 (from a speaker located in base portion 118). Though not shown in this embodiment, each earpiece 114 could alternatively include an electronic speaker (that is connected to the speakers electrically).

Referring again to FIG. 1, and as noted above, the releasably connected portion 122 is removably connected to the base portion 118 of the chest piece 110 according to this embodiment, wherein detachability of the chest piece is not essential, as noted above, but is preferred for reasons described herein. Various modes of connection between the base portion and the chest piece may be employed. For example, and according to this depicted embodiment, the rear surface 123 of the releasably connected portion 122 and a front surface 117 of the base portion 118 each include magnets that are configured to attract one another to enable releasable attachment therebetween. Alternatively, various mechanical means can be employed to releasably secure the releasably connected portion 122 to the base portion 118, such as, for example, a mechanical fastener arrangement and/or use of other types of fasteners, such as hook/loop fasteners, clips, among others.

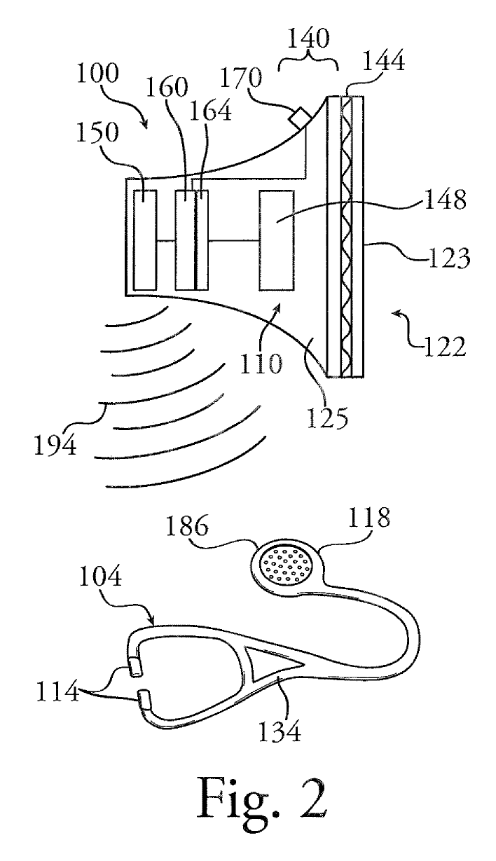

In the herein described embodiment, the stethoscopic assembly 100 further includes a microphone 140 disposed within the releasably connected portion 122 that can pick up auditory sounds. For purposes described herein, the term “microphone” refers to any suitable acoustical sensors including but not limited to traditional microphones, piezoelectric sensors, and the like. Therefore, the microphone 140 described is intended to be exemplary. Referring to FIG. 2, the microphone 140 can be located conveniently within the interior 125 of the releasably connected portion 122 and can include for example, a diaphragm 144 and a sound transducer 148 with an acoustic chamber being disposed between the transducer and diaphragm. The microphone 140 according to this embodiment is connected by known means to a transmitter 150 and a microprocessor 160, each also contained within the interior 125 of the releasably connected portion 122, as well as a control switch 170 that is disposed on the exterior of the releasably connected portion. The microprocessor 160 includes memory means 164 that is described in greater detail below.

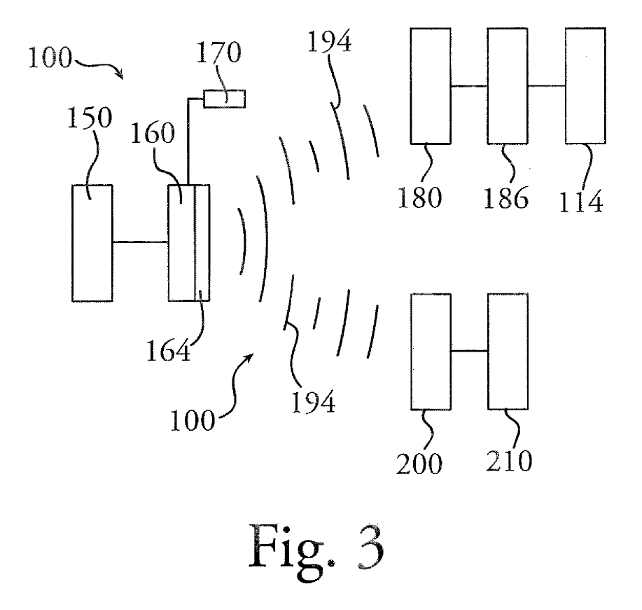

As shown schematically according to FIGS. 2 and 3, the transmitter 150 of this specific embodiment is wirelessly connected to at least one receiver 180, shown only in FIG. 3. According to this embodiment, the receiver 180 is connected to at least one speaker 186 wherein the speaker 186 is integrally provided within the base portion 118 of the chest piece 110. The location of the speaker 186, however, is optional. For example and according to another version, a speaker can be alternatively located within the yoke 134 of the headset 108 of the stethoscope 104. According to another version, an integral speaker is not present and only an external speaker is provided. An additional speaker (not shown) may be otherwise disposed as part of the stethoscopic assembly 100, such as in the earpiece 114.

The transmitter 150 may transmit sound data using any suitable method. For example, the transmitter 150 may transmit the sound data wirelessly as radio waves (RF) within a relatively small range (e.g., 5-15 feet, or other convenient range) to a plurality of multiple receivers, shown as 180. Other suitable transmission modes include infrared (IR) transmission. Preferably, the speaker 186 and the transmitter 150 are set at the same frequency. Due to the small range of the transmitter 150, or pairing method employed, multiple transmitters may operate in the same medical facility without interfering with one another. Advantageously, the transmitted signals can be heard by the wearer of the stethoscope 104, as well as other listeners in real time.

FIG. 3 is a schematic depiction of the stethoscopic assembly 100 of FIG. 1. According to this embodiment, the transmitter 150 transmits sound data to a plurality of receivers in the manner previously described when the control button 170 is activated. As in FIG. 2, some of these receivers 180 may be connected to the earpieces 114 of stethoscope 104 through a speaker 186. By providing a plurality of receivers, each of which are disposed within a corresponding number of other stethoscopic assemblies 100, sound data may be selectively shared among multiple listeners. Alternatively or in conjunction herewith, the receivers 200 may be coupled to one or more additional speakers 210, which are not specifically provided on a stethoscope that are separate, stand-alone speakers. For example, speaker 210 broadcasts the received sounds to listeners. Although FIG. 3 depicts the sound data being transmitted wirelessly, it should be appreciated, as noted above, that wired transmissions are also contemplated.

In the herein described embodiment, and referring again to FIG. 3, the microprocessor 160 of the herein described stethoscopic assembly 100 includes memory means 164 for continually buffering sound data that is picked up from the microphone 110 for a predetermined time interval. Sound data is automatically retained for a predetermined time interval (e.g., 10-15 seconds) into volatile memory wherein sound data exceeding this time interval is overwritten. The control switch 170 is connected to the microprocessor 160, the activation of the switch selectively enabling capture of buffered sound data into nonvolatile memory of the microprocessor 160 for storage, transmission and playback of captured sound data.

In the herein described embodiment and in operation, the releasably connected portion 122 is separably usable by the wearer in connection with a patient. The design of this separate component permits a physician to place the releasably connected portion 122 on the back of a patient (not shown), while the user is still facing the patient, providing considerable flexibility and versatility in use. In passing, it should be noted that this separable component can be designed according to numerous form factors and the herein described version is exemplary. As to the predetermined period of time, for example, the preceding 10 seconds of sound data may be stored in the rewritable or volatile memory of the microprocessor 160. As additional sound data is received, the oldest sound data is overwritten as noted above, thus ensuring the buffer contains the most recently received data. According to this embodiment, the user-operable control switch 170, provided on the exterior of the releasably connected portion 122 preferably is a two part switch that includes a capture control the activation of which selectively captures the sound data from the temporary buffer for storage thereof, in the memory of the microprocessor 160 and a playback control that enables the user to retrieve the sound data from storage within the microprocessor and route the sound data to the one or more receivers 180, 200 through the transmitter 150. Alternately, two separate controls can be used to capture and transmit. Such a configuration permits the user to hear a sound of interest in real time and thereafter record such sound to the storage location. The user may then replay the sound for the benefit of the patient or other listeners. The user-operable control switch 170 may be provided on the releasably connected portion 122 as in the present embodiment or alternatively, the switch can be disposed on the user-worn chest piece 110, or on another suitable location. Similarly, the microprocessor 160, including the rewritable memory and storage, may be located within the releasably connected portion 122, the user-worn chest piece 110, or another suitable location in which the user has access to a control to permit record and playback selectively.

The user may play sounds other than the captured sound data. For example, a prerecorded sound may be selected from a digital library of sounds that includes examples of both normal and abnormal sounds. Such a configuration permits the user to play the captured sound data and thereafter play a sample “normal” sound clip. This enables the listener to compare the captured sound to a healthy sound and helps facilitate doctor-patient communication. Additionally or alternatively, the captured sound may be compared to an abnormal sound clip so the patient can hear the similarities.

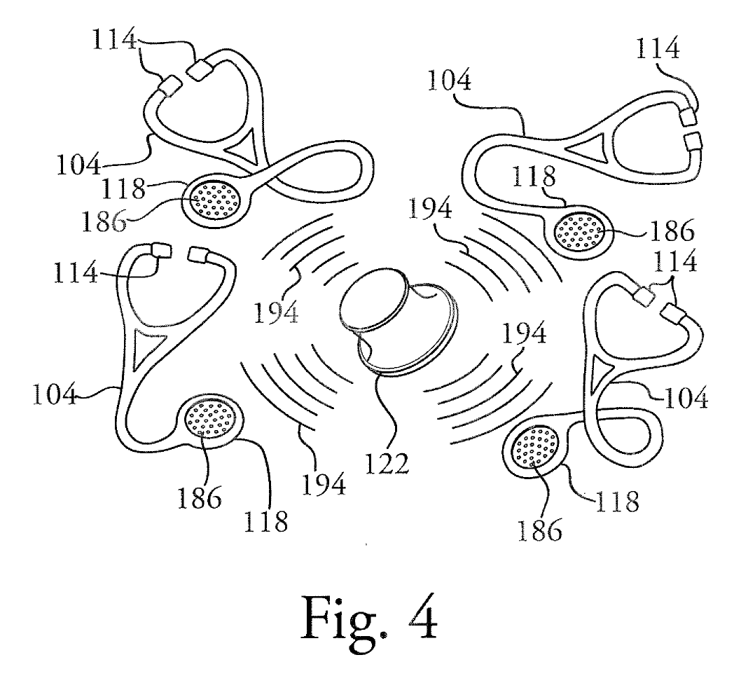

FIG. 4 illustrates the transmission of sound data from the releasably connected portion 122 of the stethoscopic assembly to a plurality of stethoscopes 104, each of which includes a speaker 186 disposed in the base portion 118 of the chest piece. Activation of the control switch 170 enables captured sound data to be heard by the wearers of the stethoscopes 104 through the ear plugs 114 and also to other interested parties through the speakers 186.

### PARTS LIST FOR FIGS. 1-4

100 stethoscopic assembly
104 stethoscope
108 headset
110 chest piece
114 earpieces
117 rear surface, base portion
118 base portion
122 releasably connected portion
123 front surface, releasably connected portion
125 interior
126 hoses
130 single hose
134 yoke
140 microphone
144 diaphragm
148 transducer
150 transmitter
160 microprocessor
164 memory means
170 control switch
180 receiver
186 speaker
194 sound data
200 receiver
210 speaker

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

What is claimed is:

1. A stethoscope assembly, said assembly comprising:
a stethoscope including a chest piece that is interconnected to at least one earpiece, said chest piece including an acoustical sensor enabling sounds from a patient to be heard through said at least one earpiece, said assembly further including:
a transmitter;
a receiver enabling reception of signals from said transmitter wherein said receiver enables a wearer of said stethoscope to hear received sounds through said at least one earpiece; and
memory means that continually buffers sound data picked up from said microphone for a predetermined time period and at least one user-operable control switch connected to said memory means and said transmitter for enabling capture of sound data for storage, transmission and playback of said captured sound data.

2. The stethoscope assembly as recited in claim 1, including at least one speaker that selectively broadcasts said received sounds.

3. The stethoscope assembly as recited in claim 1, wherein said chest piece includes a releasably connected portion that includes said acoustical sensor and said transmitter.

4. The stethoscope assembly as recited in claim 3, wherein said transmitter and receiver are wirelessly interconnected.

5. The stethoscope assembly as recited in claim 1, wherein said at least one earpiece includes an electronic speaker.

6. The stethoscope assembly as recited in claim 2, wherein sound data are transmitted in real time via said receiver to said at least one earpiece and in which actuation of said at least one control switch causes captured sound data to be routed to said at least one speaker for broadcast.

7. The stethoscope assembly as recited in claim 2, wherein said at least one speaker is integral to said chest piece.

8. The stethoscope assembly as recited in claim 2, wherein said at least one speaker is not provided on said stethoscope.

9. The stethoscope assembly as recited in claim 3, wherein said releasably connected portion is magnetically attached to said chest piece.

10. The stethoscope assembly as recited in claim 3, wherein said releasably connected portion is mechanically attached to said chest piece.

11. The stethoscope assembly as recited in claim 3, wherein said at least one user-operable control switch is disposed in said releasably connected portion.

12. The stethoscope assembly as recited in claim 11, wherein said at least one user-operable control switch includes a capture control that captures said sound data and stores said data in said memory means and further includes a playback control.

13. The stethoscope assembly as recited in claim 1, further comprising a digital library of prerecorded sounds.

14. A method for transmitting an auditory signal from a patient to a receiver using a stethoscope, said stethoscope including at least one earpiece interconnected to a chest piece, said chest piece having an acoustical sensor to detect sounds to be heard through said at least one earpiece, said method comprising the steps of:
releasing a portion of said chest piece and placing said released portion in relation to a patient, said released portion including said acoustical sensor and a wireless transmitter, wherein said wireless transmitter transmits said sound data, said chest piece including a first wireless receiver;
listening to sounds produced by said patient wherein sounds are transmitted in real time to said first wireless receiver from said wireless transmitter to enable a wearer of said stethoscope to hear said sound data through said at least one earpiece;
continually buffering sound data picked up by said acoustical sensor; and
selectively capturing buffered sound data for broadcast through at least one speaker, enabling the user of said stethoscope and at least one other person to hear selected sounds from said patient.

15. The method as recited in claim 14, further comprising the step of storing the buffered sound data in a storage location, said method further comprising the step of selectively playing back the captured buffered sound data through an integral speaker of said stethoscope.

16. The method as recited in claim 15, further comprising the step of broadcasting said captured buffered sound data from the storage location through said at least one speaker.

17. The method as recited in claim 14, wherein the sound data is buffered for a predetermined time period that is less than about fifteen seconds.

18. The method as recited in claim 14, further comprising the step of storing the buffered sound data in a storage location in the releasable section of the chest piece.

19. The method as recited in claim 14, further comprising the step of storing the buffered sound data in a storage location in the user-worn section of the chest piece.

20. A stethoscope assembly, said assembly comprising:
a binaural stethoscope including a chest piece, a pair of earpieces interconnected to said chest piece, a memory buffer enabling sounds to be stored, said chest piece including an acoustical sensor enabling sounds of a patient to be heard through said earpieces, said chest piece including a releasably connected portion including said acoustical sensor;
a wireless transmitter disposed within said releasably connected portion;
a first wireless receiver enabling reception of a transmitted sound from said wireless transmitter;
a first speaker, integral with said chest piece, that enables routing of received sounds from said first wireless receiver such that a wearer of said stethoscope may hear said transmitted sound in real time through said earpieces; and
at least one additional speaker that enables a listener to hear said received sound from said at least one additional speaker.

21. The stethoscope assembly as recited in claim 20, wherein said at least one additional speaker is provided on a separate stethoscope.

22. A method for selectively transmitting an auditory signal from a patient to at least one receiver using a stethoscope, said stethoscope including at least one earpiece interconnected to a chest piece, said chest piece having an acoustical sensor to detect sounds to be heard through said at least one earpiece, said method comprising the steps of:
placing said chest piece in relation to said patient and picking up sounds of said patient with said acoustical sensor;
listening to sounds produced by said patient wherein sounds are transmitted in real time to said at least one earpiece;
continually buffering sound data picked up by said acoustical sensor; and
selectively capturing buffered sound data for broadcast through at least one speaker, enabling the user of said stethoscope and at least one other person to hear selected sounds from said patient.

23. The method as recited in claim 22, further comprising the step of broadcasting a prerecorded sound from a library of sounds through said at least one speaker.

24. The method as recited in claim 22, further comprising the step of storing the buffered sound data in a storage location, said method further comprising the step of selectively playing back the captured buffered sound data through said at least one speaker.

25. The method as recited in claim 22, further comprising the step of broadcasting said captured buffered sound data from the storage location through said at least one speaker.

26. The method as recited in claim 22, wherein the sound data is buffered for a predetermined time period that is less than about fifteen seconds.

27. The method as recited in claim 22, further comprising the step of storing the buffered sound data in a storage location in the releasable section of the chest piece.

28. The method as recited in claim 22, further comprising the step of storing the buffered sound data in a storage location in the user-worn section of the chest piece.
